(19) United States
(12) Patent Application Publication
(10) Pub. No.: US 2025/0221827 A1
(43) Pub. Date: Jul. 10, 2025

(54) SPINE PROCESS SUPPORTING ELEMENT

(71) Applicant: Socko Medical Co., Ltd., New Taipei (TW)

(72) Inventors: JUNG-TSOU TUNG, New Taipei (TW); HAO-YUN TUNG, New Taipei (TW); CHANG-YI KUO, New Taipei (TW)

(21) Appl. No.: 18/665,662

(22) Filed: May 16, 2024

(30) Foreign Application Priority Data
Jan. 5, 2024 (TW) 113100531

(51) Int. Cl. A61F 2/44

(52) U.S. Cl. CPC A61F 2/442; A61F 2002/443

(57) ABSTRACT

A spine process supporting element includes two first side surfaces and two second side surfaces. The two first side surfaces are symmetrically provided on the two opposite sides of an axis, each first side surface includes a ratchet surface, the two opposite ends of the ratchet surface parallel to the axis are respectively arcuately extended toward the axis and the width thereof is gradually decreased to form a first head tapered arc surface and a first tail tapered arc surface; two second side surfaces; the two second side surface are symmetrically provided on the opposite sides of the axis, each second side surface includes a connection surface the two opposite ends of the connection surface parallel to the axis are respectively arcuately extended toward the axis and the width thereof is gradually decreased to form a second head tapered arc surface and a second tail tapered arc surface.

### CROSS REFERENCE TO RELATED APPLICATION

All related applications are incorporated by reference. The present application is based on, and claims priority from, Taiwan Application Serial Number 113100531, filed on Jan. 5, 2024, the disclosure of which is hereby incorporated by reference herein in its entirety.

### TECHNICAL FIELD

The present invention relates to the field of medical technology, and in particular to a spine process supporting element.

### BACKGROUND

For patients with spinal diseases, it is easy to produce pathological atrophy in the intervertebral disc, causing the spinal gap to shrink and compress the spinal nerves, causing paralysis or pain.

In order to solve the above problems, conventional treatment methods include implanting supports, pedicle screws or artificial intervertebral disc fillers, all of which aim to elevate the vertebrae and open the neural foramen gap so that the vertebrae are in a normal position, and at the same time, an appropriate distance can be maintained between the two vertebrae, so that the vertebrae will not atrophy or deform, thereby reducing or eliminating the patient's discomfort.

As far as the currently used supports are concerned, some supports must destroy the spinal ligaments during installation, and there are common defects such as complex structures, difficult operations, difficulties in installation and removal, etc., resulting in the need for long-term operations and an increase in the number of patients' risks of bleeding and infection.

Although other supports claim to have improved structures, which are beneficial to inserting the support between the vertebrae, the structure of this type of conventional support has many sharp edges and corners, making it not only difficult to insert the support between the atrophied vertebrae, but also when placed between the vertebrae, or when moving or rotating between the vertebrae to adjust the position of the support, it is easy to scratch the surface of the spine and cause secondary damage to the spine.

In addition, this type of conventional support element is provided with many fine teeth, whose function is originally to increase the bite force between the support element and the spine. However, such fine teeth will wear out after long-term contact with the spine, causing the support member to shift, affecting the support of the spine.

Furthermore, the operator must use special tools to hold the support element in order to accurately place the support element between the vertebrae, which is not only costly but also inconvenient to operate.

Regarding the material of the conventional support element, the commonly used materials are mainly divided into metal (for example, titanium alloy, tantalum, cobalt-chromium-molybdenum alloy) and polymer polyetheretherketone (for example, PEEK) composite materials. However, because the material is hydrophobic and biologically inert, it lacks an osteoinductive mechanism and cannot actively interact with natural bone tissue, which is not conducive to the adhesion and growth of bone cells and prevents the implant from being tightly integrated with bone tissue. Therefore, the osseointegration ability is poor, resulting in poor osseointegration and prolonged patient recovery period.

Based on this, how to develop a device that has a unique structural design, is easy to implant and fit with the spine, can improve operational convenience, can be used with different tools without being limited to a single tool, and its material has been specially treated to improve the affinity between bone cells and implants, the promotion of osseointegration, the restoration of the stability of spinal joints, and the “intervertebral support elements” that reduce the patient's recovery period are issues that people in the relevant technical field urgently need to solve.

### SUMMARY

The present invention proposes a spine process supporting element, including: two first side surfaces, symmetrical about an axis and respectively provided on two opposite sides of the axis, each of the first side surfaces including a ratchet surface, the two ratchet surface spaced a distance and parallel to an XY plane formed by an X-axis and a Y-axis, the axis parallel to the X-axis, two ends of the ratchet surface parallel to the axis respectively arcuately extended toward the axis and the width thereof gradually decreased to form a head tapered arc surface and a first tail tapered arc surface, a plurality of unidirectional ratchets provided on the ratchet surface, and a tip of each of the unidirectional ratchets facing the first tail tapered arc surface; and two second side surfaces, symmetrical about the axis and respectively provided on two opposite sides of the axis, each of the second side surfaces including a connection surface, the two connection surface spaced a distance and parallel to a n XZ plane formed by the X-axis and a Z-axis, the X-axis, the Y-axis and the Z-axis being three axes perpendicular to one another, two opposite ends of the connection surface parallel to the axis respectively arcuately extended toward the axis and the width thereof gradually decreased to form a second head tapered arc surface and a second tail tapered arc surface; the two second side surfaces positioned between the two first side surfaces, each of the connection surfaces parallel to two corresponding sides of the axis and respectively connected to an edge of the ratchet surface, the two first head tapered arc surfaces and the two second head tapered arc surface spaced apart and connected to each other to form an conical tapered head, the center of the head positioned on the axis, the two first tail tapered arc surfaces and the two second tail tapered arc surfaces separated apart and connected to each other to form a tail, the tail provided with a connection hole, and the center of the connection hole coaxial with the axis.

### BRIEF DESCRIPTION OF THE DRAWINGS

FIG. 1 is a perspective view of the structures of a front side, right side and top side of an embodiment of the present invention;

FIG. 2 is a perspective view of the structures of a rear side, left side and bottom side of the embodiment of FIG. 1;

FIG. 3 is a front view of the embodiment of FIG. 1;

FIG. 4 is a back view of the embodiment of FIG. 1;

FIG. 5 is a right-side view of the embodiment of FIG. 1;

FIG. 6 is a top view of the embodiment of FIG. 1;

FIGS. 7 and 8 respectively are a perspective of the embodiment of FIG. 1 matching with a tool;

FIGS. 9 to 12 respectively are a schematic view of a continuous movement of the embodiment of FIG. 1 installed between spines with the tool;

FIG. 13 is a perspective view of the embodiment of FIG. 1 matching with a tool from another viewing angle; and

FIGS. 14 to 16 respectively are a perspective view of a continuous movement of the embodiment of FIG. 11 installed between spines with the tool.

### DETAILED DESCRIPTION

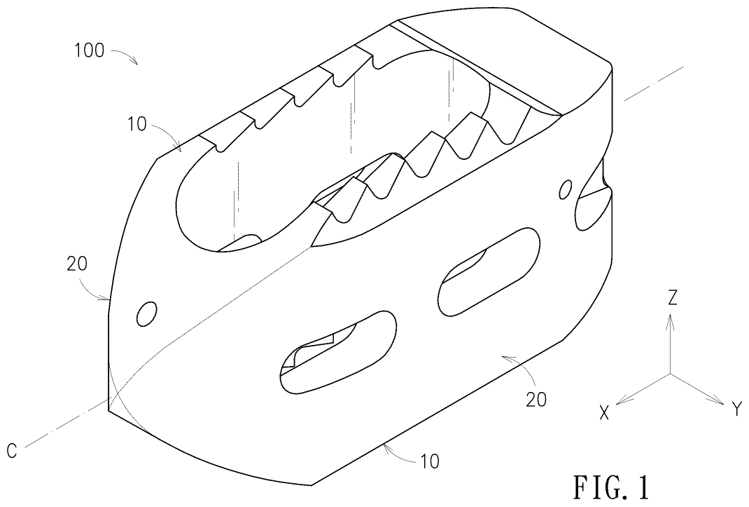

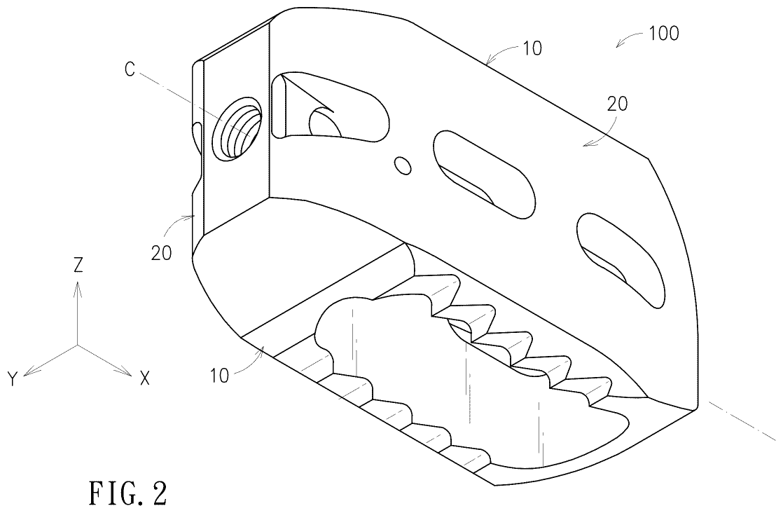

Referring to FIGS. 1 and 2, a spine process supporting element 100 of the present invention includes two first side surfaces 10 arranged on the upper and lower, opposite to each other and symmetrical about an axis C, and two second side surfaces 20 arranged on the right and the left, opposite to each other and symmetrical about the axis C.

The material of the spine process supporting element 100 is not limited, for example, it can be biocompatible metal or plastics, and the material of the spine process supporting element 100 is sandblasted, large grit, acid-etched (SLA), thereby promoting bone cell affinity and growth, and increasing bone graft fusion to shorten bone healing time.

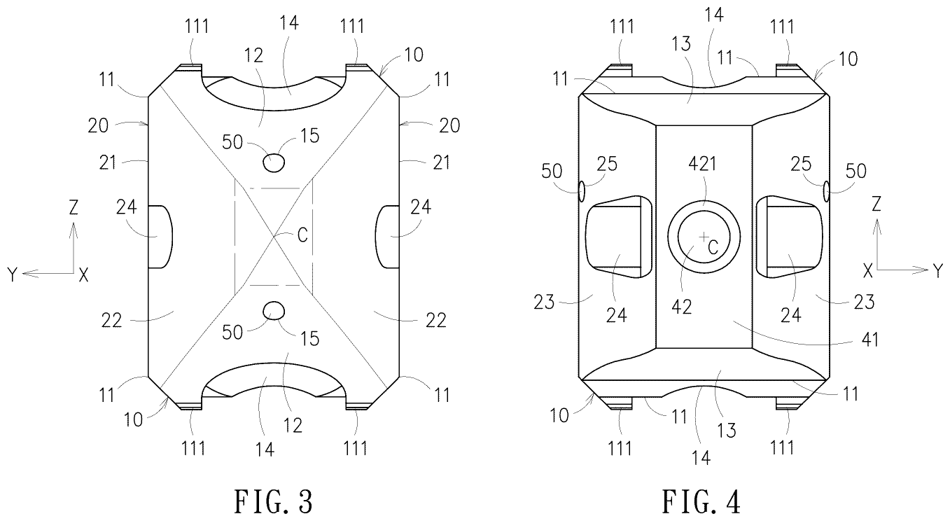

Referring to FIGS. 3 to 6, the two first side surfaces 10 are symmetrical about the axis C, and respectively provided on the two opposite sides of the axis C. Each of the first side surfaces 10 includes a ratchet surface 11, and the two ratchet surfaces 11 separated a distance and parallel to a XY plane formed by an X-axis and a Y-axis. The axis C is parallel to the X-axis.

The two opposite ends of the ratchet surface 11 are parallel to the axis C, extended arcuately toward the axis C respectively and have tapered widths to form a first head tapered arc surface 12 and a first tail tapered arc surface 13.

A plurality of unidirectional ratchets 111 are provided on the rachet surface 11, and a tip 112 of each of the unidirectional rachets 111 faces the first tail tapered arc surface 13.

Referring to FIGS. 3 to 6 again, the two second side surfaces 20 are symmetrical to the axis C and are respectively provided on the two opposite sides of the axis C. Each of the second side surfaces 20 includes a connection surface 21, and the two connection surfaces 21 spaced apart and parallel to an XZ plane formed by the X-axis and a Z-axis, and the X-axis, the Y-axis and the Z-axis are three axes perpendicular to one another.

The two opposite ends of the connection surface 21 are parallel to the axis C, extended arcuately toward the axis C respectively, and have tapered widths to form a second head tapered arc surface 22 and a second tail tapered arc surface 23.

The second side surfaces 20 are positioned between the first side surfaces 10. The two opposite sides of each of the connection surfaces 21 parallel to the axis Care respectively connected to the edge of a ratchet surface 11.

The two first head tapered arc surfaces 12 and the two second head tapered arc surfaces 22 are spaced apart and connected to each other to form a circular arc pointed conical head 30, the center of which is positioned at the axis C.

The two first tail tapered arc surfaces 13 and the two second tail tapered arc surfaces 23 are spaced apart and connected to each other to form a tail 40.

Referring to FIGS. 4 to 6 again, the tail 40 has a plane 41, which is parallel to a YZ plane formed by the Y-axis and the Z-axis.

The plane 41 and the head 30 are two opposite ends of the spine process supporting element 100, where a connection hole 42 with inner threads 421 is provided on the plane 41 of the tail 40, and the center of the connection hole 42 is coaxial with the axis C.

Referring to FIGS. 3 to 6 again, each of the first side surfaces 10 is provided with a first hole 14 passed through the first side surface 10. The two sides of each of the first hole 14 parallel to the axis C are respectively provided with a plurality of unidirectional ratchets 111.

Each of the second side surfaces 20 is provided with three second holes 24 passed through the second side surface 20. Among them, two second holes 24 are provided on each of the connection surfaces 21, and one second hole 24 is provided on the second tail tapered arc surface 23.

Each of the first holes 14 is in communication with each of the second holes 24.

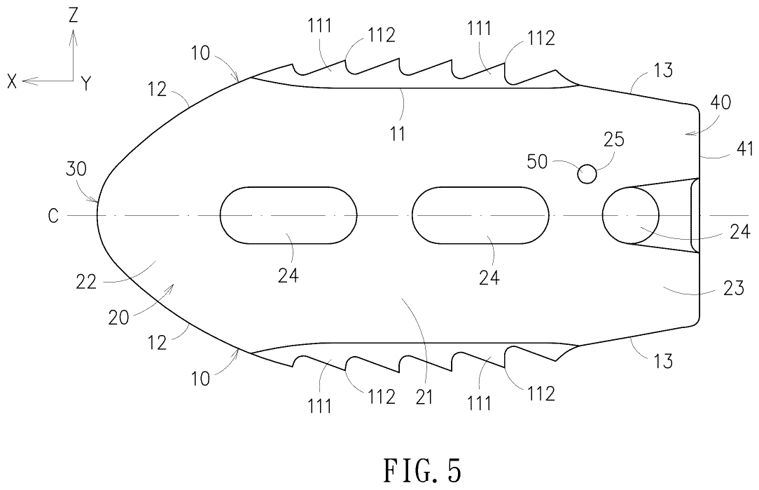

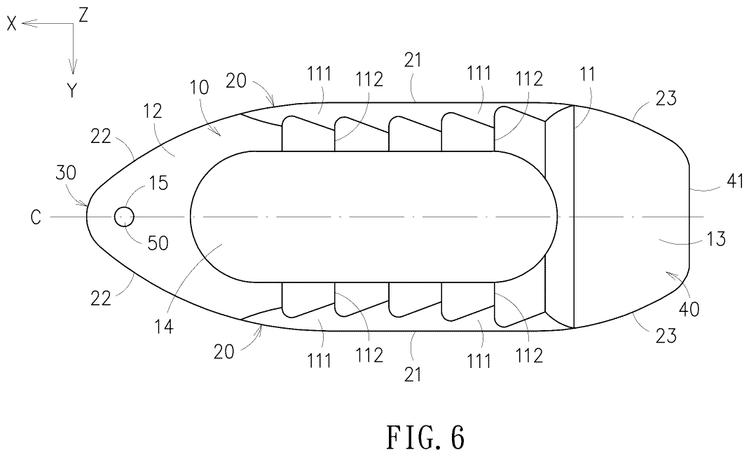

FIGS. 5 and 6 show that the first hole 14 and the second hole 24 are oblong, but they are not limited to this. They can be designed according to actual needs, and the shape and quantity are not limited.

Referring to FIGS. 3 to 6, a first concave hole 15 is provided at the corresponding position of the first head tapered arc surface 12 of the two first side surfaces 10. A second concave hole 25 is provided at a corresponding position of the second tail tapered arc surface 23 of the two second side surfaces 20.

The first concave hole 15 and the second concave hole 25 can be used to embed metal 50 for imaging during the operation, for example, tantalum alloy, titanium alloy, pure titanium, which is convenient to perform postoperative imaging to track the position of the spine process supporting element 100 and observe the fusion between the spine process supporting element 100 and the spine.

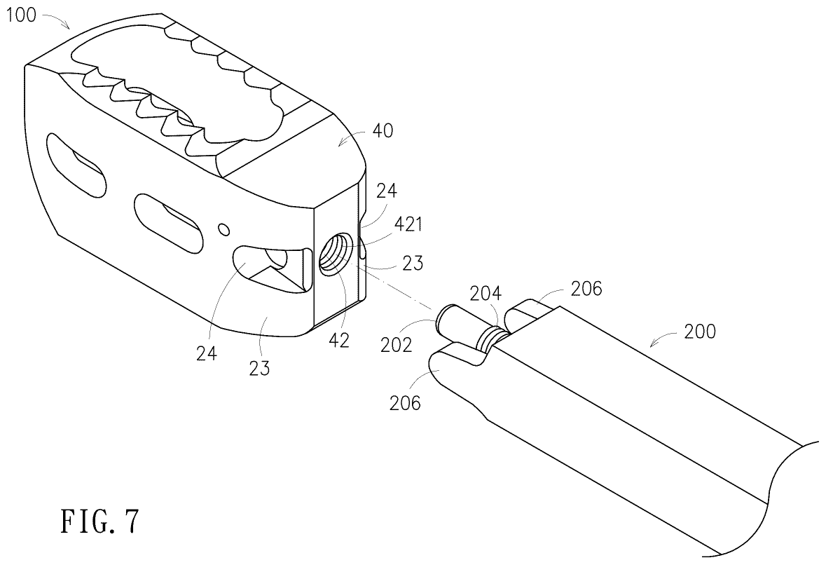

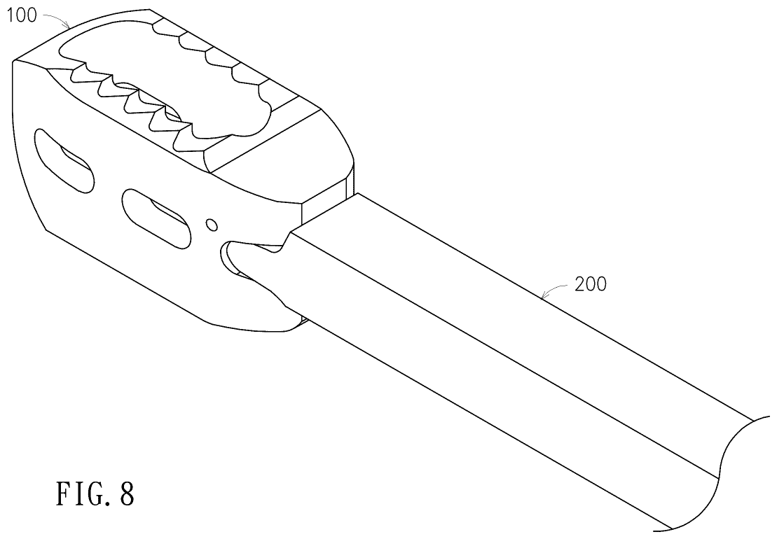

Referring to FIGS. 7 and 8, the connection hole 42 with the inner threads 421 provided on the tail 40 of the spine process supporting element 100 can provide connection with a tool 200, where the tool 200 has a connection column 202 with outer threads 204, where the two sides of the connection column 202 are respectively provided with a convex block 206. The connection column 202 is screwed in the connection hole 42, and the convex block 206 is embedded in the second hole 24 provided on the corresponding position of the second tail tapered arc surface 23, and the tool 200 can then be allowed to be connected to the spine process supporting element 100.

It needs to be noted that not limited to the tool 200 shown in FIG. 7, any tool that can be screwed into the connection hole 42 is suitable for the spine process supporting element 100. In addition, the connection structure between the connection hole and the tool is also not limited to threads, for example, it can be a buckle, a hook, or other structures, or it can be locked with bolts.

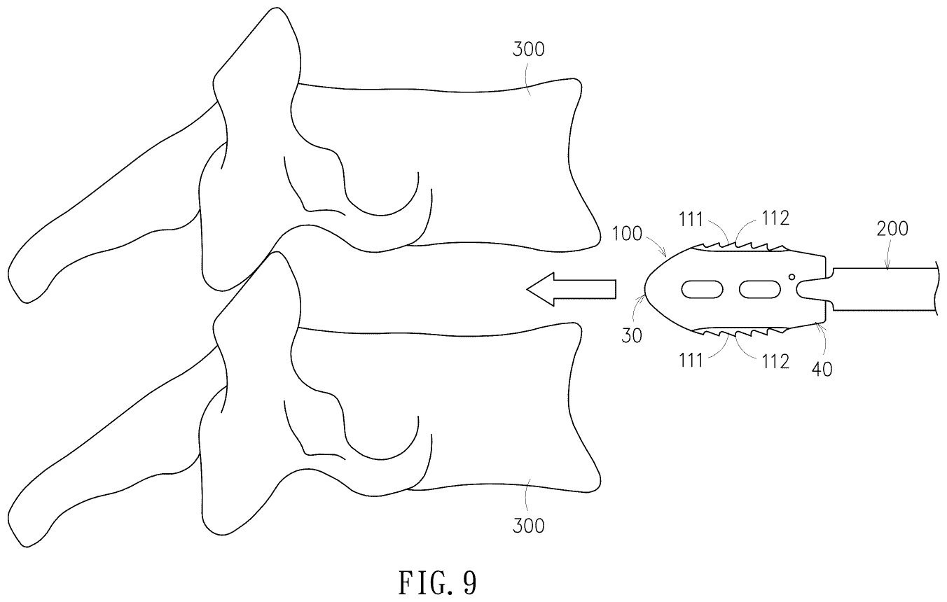

Referring to FIGS. 9 to 11, the figures illustrate the continuous movement of the spine process supporting element 100 and the tool 200 installed between spines 300.

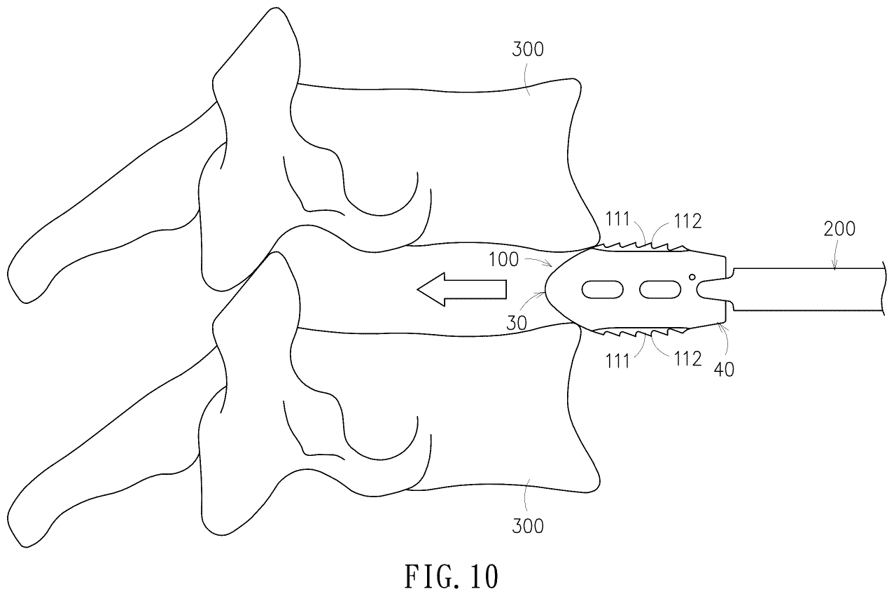

Referring to FIGS. 9 and 10 again, an operator (for example, a doctor) uses the tool 200 to insert the spine process supporting element 100 into the space between the two spines 300. The arc tapered head 30 is conducive to the spine process supporting element 100 to spread the two spines 300 apart. Furthermore, since the tip 112 of the unidirectional ratchet 111 faces the tail 40, it is advantageous for the spine process supporting element 100 to extend into the space between the two spines 300.

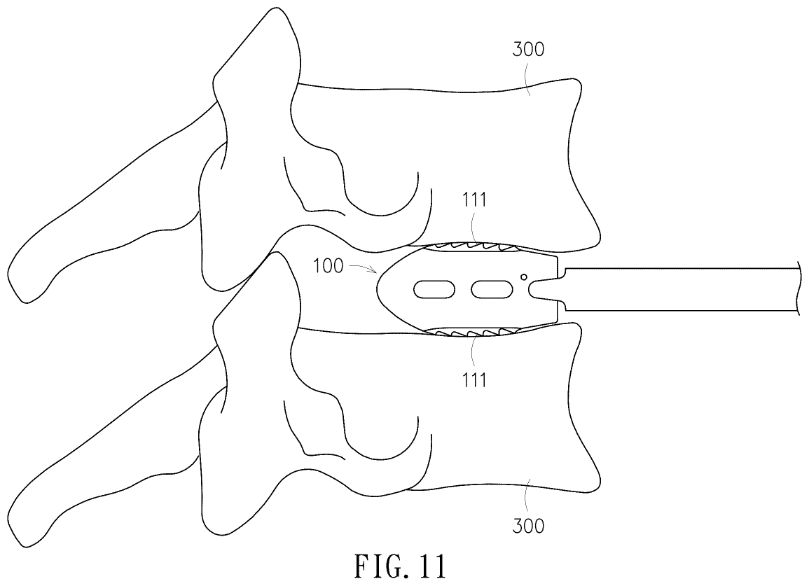

Referring to FIG. 11, after the spine process supporting element 100 is placed in the space between the two spines 300, the unidirectional ratchet 111 is used to engage with the surfaces of the spines, capable of preventing the spine process supporting element 100 from sliding.

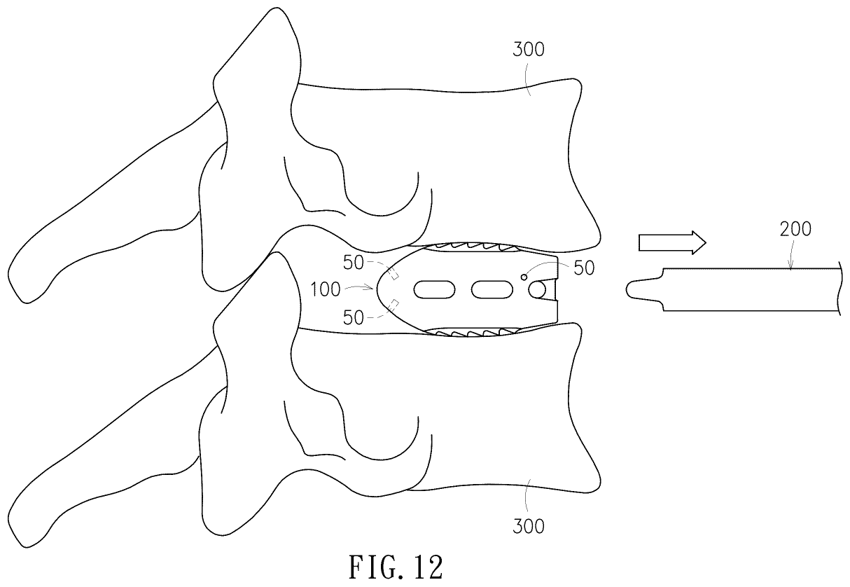

Referring to FIG. 12, after confirming that the spine process supporting element 100 is positioned, the operator can then separate the tool 200 from the spine process supporting element 100, allowing the spine process supporting element 100 to be stayed in the space between the two spines 300, and the spine process supporting element 100 is used to support the two spines with an appropriate distance. Furthermore, after surgery, the metal 50 for imaging can be used to track the position of the spine process supporting element 100 and observe the fusion of the spine process supporting element 100 and the spines 300.

Furthermore, the material of the spine process supporting element 100 is sandblasted, large grit, acid-etched (SLA), which can promote the affinity and growth of bone cells, improve bone graft fusion, and shorten the bone healing time.

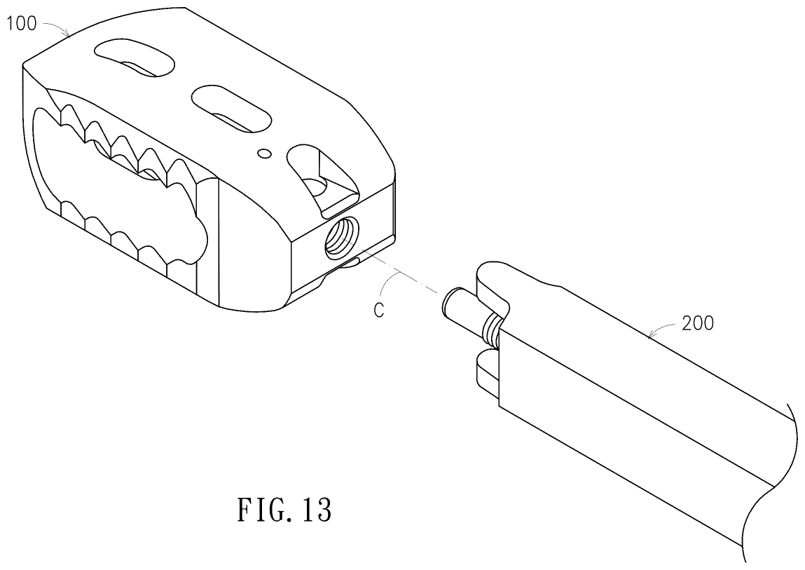

Referring to FIG. 13, the connection manner of the spine process supporting element 100 and the tool 200 is the same as the aforementioned, but the spine process supporting element 100 and the tool 200 are turned 90 degrees around the axis C, and can also be inserted into the space between the spines in this manner.

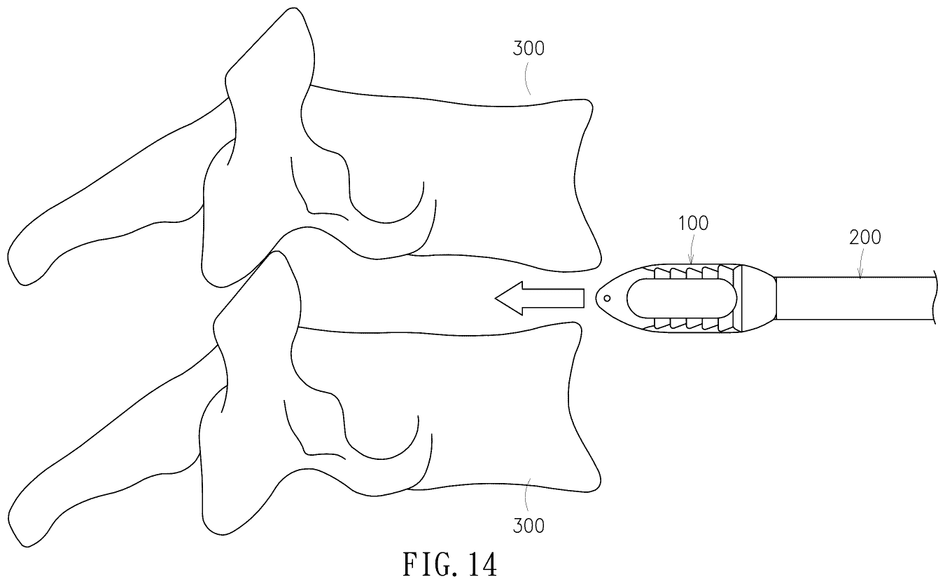

Referring to FIGS. 14 to 16, the figures illustrate the continuous movement of installing the spine process supporting element 100 with the tool 200 between the spines 300 in the arrangement shown in FIG. 13.

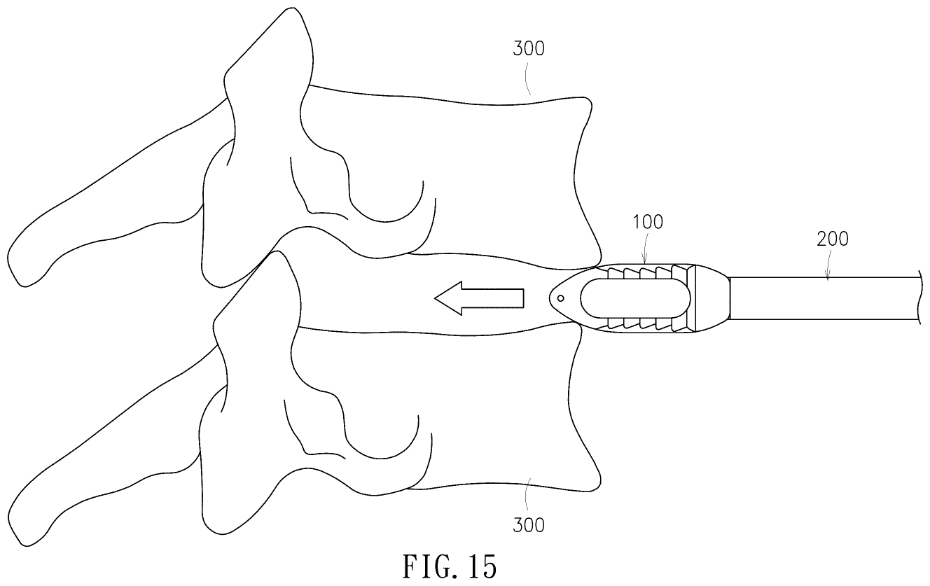

Referring to FIGS. 14 and 15 again, the operator (for example, a doctor) uses the tool 200 to insert the spine process supporting element 100 into the space between the two spines 300. The conical tapered head 30 facilitates the spine process supporting element 100 to spread the two spines 300 apart.

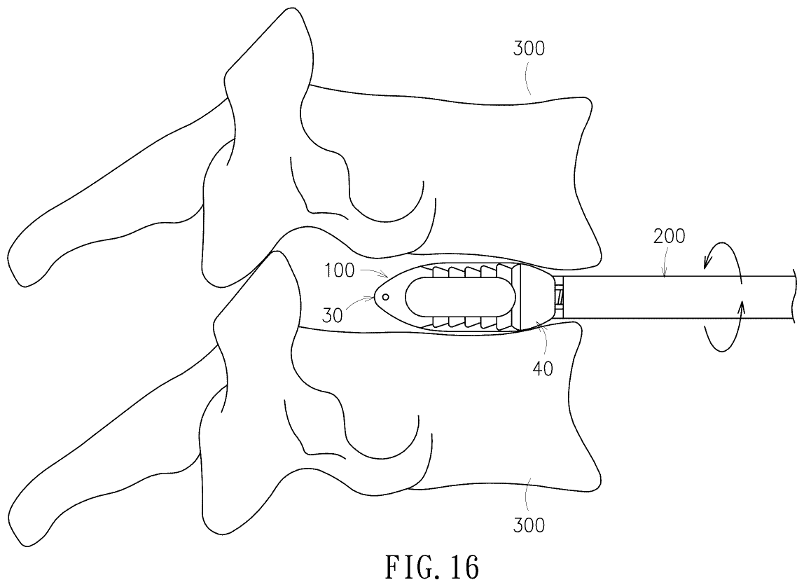

Referring to FIG. 16, after the spine process supporting element 100 is placed in the space between the two spines 300, the operator uses the tool 200 to drive the spine process supporting element 100 to rotate 90 degrees. Since the head 30 and the tail 40 respectively have a conical tapered design, it is advantageous for the spine process supporting element 100 to rotate between the two spines 300. Thereafter, the spine process supporting element 100 is allowed to form the state shown in FIG. 11. The unidirectional ratchet 111 is used to engage with the surfaces of the spines 300, which can prevent the spine process supporting element 100 from sliding.

Thereafter, as shown in FIG. 12, after confirming that the spine process supporting element 100 is positioned, the operator can then separate the tool 200 from the spine process supporting element 100, allowing the spine process supporting element 100 to be stayed between the two spines 300; the spine process supporting element 100 is used to support the two spines 300 with an appropriate distance. Furthermore, after surgery, the metal 50 for imaging can be used to track the position of the spine process supporting element 100 and observe the fusion of the spine process supporting element 100 and the spines 300.

Similarly, the material of the spine process supporting element 100 is sandblasted, large grit, acid-etched (SLA), which can promote the affinity and growth of bone cells, improve bone graft fusion, and shorten the bone healing time.

In summary, both the head and the tail of the spine process supporting element disclosed by the present invention are formed by four convex arc surfaces. Especially, the head is a conical tapered body, easy to implant and rotate between the spines; the two opposite surfaces have large barb-type ratchets, improving the engagement force between the spine process supporting element and the spines; the tail is provided with the connection hole and insertion grove, capable of being used with different types of tools and not limited to a single type of tool; the material has been treated with special sandblasted, large grit, acid-etched (SLA) process to create an environment suitable for bone cells to adhere to, which can improve the affinity between bone cells and implants and promote osseointegration, restore the stability of spinal joints and reduce the patient's recovery period; there is a concave hole for burying the metal used for imaging during surgery, so that it can be easily visualized after surgery to track the position and observe its fusion with the spine.

Although the present invention has been disclosed above through embodiments, it is not intended to limit the present invention. Anyone with ordinary knowledge in the technical field can make some modifications without departing from the spirit and scope of the present invention. and modifications, so the protection scope of the present invention shall be determined by the appended claims.

What is claimed is:

1. A spine process supporting element, comprising:
two first side surfaces, symmetrical about an axis and respectively provided on two opposite sides of said axis, each of said first side surfaces comprising a ratchet surface, said two ratchet surface spaced a distance and parallel to an XY plane formed by an X-axis and a Y-axis, said axis parallel to said X-axis, two ends of said ratchet surface parallel to said axis respectively arcuately extended toward said axis and the width thereof gradually decreased to form a head tapered arc surface and a first tail tapered arc surface, a plurality of unidirectional ratchets provided on said ratchet surface, and a tip of each of said unidirectional ratchets facing said first tail tapered arc surface; and
two second side surfaces, symmetrical about said axis and respectively provided on two opposite sides of said axis, each of said second side surfaces comprising a connection surface, said two connection surface spaced a distance and parallel to an XZ plane formed by said X-axis and a Z-axis, said X-axis, said Y-axis and said Z-axis being three axes perpendicular to one another, two opposite ends of said connection surface parallel to said axis respectively arcuately extended toward said axis and the width thereof gradually decreased to form a second head tapered arc surface and a second tail tapered arc surface;
said two second side surfaces positioned between said two first side surfaces, each of said connection surfaces parallel to two corresponding sides of said axis and respectively connected to an edge of said ratchet surface, said two first head tapered arc surfaces and said two second head tapered arc surface spaced apart and connected to each other to form an conical tapered head, the center of said head positioned on said axis, said two first tail tapered arc surfaces and said two second tail tapered arc surfaces separated apart and connected to each other to form a tail, said tail provided with a connection hole, and the center of said connection hole coaxial with said axis.

2. The element according to claim 1, wherein each of said first sides is provided with at least one first hole penetrating each of said first side surfaces and each of said second side surfaces is provided with at least one second hole penetrating each of said second side surfaces, and each of said first holes and each of said second holes are in communication with each other.

3. The element according to claim 2, wherein two sides of each of said first holes parallel to said axis are respectively with a plurality of said unidirectional ratchets.

4. The element according to claim 2, wherein each of said connection surfaces is provided with said two second holes, and said second tail tapered arc surfaces is provided with one of said second holes.

5. The element according to claim 1, wherein said connection hole has inner threads.

6. The element according to claim 1, wherein said spine process supporting element is made of biocompatible metal or plastic and is sandblasted, large grit, Acid-etched (SLA).

7. The element according to claim 1, wherein a first concave hole is provided at a corresponding position of said two first side surfaces, and a second concave hole is provided at a corresponding position of said two second side surfaces for burying the metal for development.

8. The element according to claim 7, wherein each of said first concave holes is provided on said first head tapered arc surface, and each of said second concave hole is provided on said second tail tapered arc surface.

9. The element according to claim 7, wherein said metal for development is tantalum alloy, titanium alloy, or pure titanium.

10. The element according to claim 1, wherein said tail has a plane parallel to said YZ formed by said Y-axis and said Z-axis, and said plane and said head are opposite ends of said spine process supporting element, and said connection hole is located on said plane.
